(19) United States
(12) Patent Application Publication
(10) Pub. No.: US 2025/0331711 A1
(43) Pub. Date: Oct. 30, 2025

(54) OPTICAL SYSTEM FOR VISUAL FIELD TESTING

(71) Applicant: XENON OPHTHALMICS INC., Belleville, NJ (US)

(72) Inventors: ZESHAN ALI KHAN, BELLEVILLE, NJ (US); STEVE SUSANIBAR, Morris Plains, NJ (US); Viachaslau LOSIK, Hemau (DK); Siarhei SIDAROVICH, Novoselye, Minsk district (BY)

(73) Assignee: XENON OPHTHALMICS INC., Belleville, NJ (US)

(21) Appl. No.: 18/855,401

(22) PCT Filed: Apr. 11, 2023
(86) PCT No.: PCT/US2023/018209

§ 371 Date: Oct. 9, 2024

Related U.S. Application Data
(60) Provisional application No. 63/362,866, filed on Apr. 12, 2022.

(51) Int. Cl. A61B 3/024; A61B 3/00; A61B 3/028; A61B 3/113

(52) U.S. Cl. CPC A61B 3/024; A61B 3/0008; A61B 3/113; A61B 3/0285

(57) ABSTRACT

An optical system comprises a pair of matching optical assemblies mounted within a housing that is attachable to a VR style headset frame. Each optical assembly has an eyepiece followed by a beamsplitter leading to an IR eye tracking channel and a display channel with a visual display and a diopter adjustable lens assembly to allow the apparent distance of an image shown on the display to be varied. The optical system can be used for visual field testing, as a mechanical phoropter replacement, or for use in other optical testing and viewing applications.

### CROSS-REFERENCE TO RELATED APPLICATIONS

This application claims priority to U.S. Provisional Patent Application No. 63/362,866, filed Apr. 12, 2022, the entire contents of which is incorporated by reference.

### FIELD OF THE INVENTION

The present invention is related to improvements in systems for visual testing and imaging.

### BACKGROUND

Eye care is an important part of overall health and many specialized systems have been developed to allow ophthalmologists to examine a person's eyes. Many of these devices are expensive, limiting their availability. They are also bulky, often requiring a dedicated table or mounting in a special stand. The size, weight, and general ungainliness of these devices also can require dedicated space in a doctor's office to be reserved for that equipment. For example, a mechanical phoropter is the default standard for determining the proper prescription for a patient. While accurate, these devices are largely installed in-location and cannot be easily moved.

Patients who have mobility limitations may not be able to easily make an office visit or be physically able to position themselves as required for examination using a particular optical tool. This can limit the ability to provide comprehensive eye examinations to these patients. Likewise, due to bulk and expense, it may be difficult or impossible to bring a variety of these specialized eye examination systems to a patient who is not able to travel to the doctor's office.

It is known to provide certain types of eye testing equipment in a portable form that can be integrated into a head mounted system. However, these systems can be limited. There is a need for a portable and inexpensive system for performing eye examinations in a variety of environments, that can easily be used for visual field and prescription testing, and that has an architecture that is well suited for use in a headset mounted implementation.

### SUMMARY

An improved optical assembly suitable for integration a VR-style headset mount and that addresses the deficiencies with prior systems is disclosed herein.

In an embodiment, the system comprises an optical module that can be integrated with or removal attached to a headset frame configured to be worn on the face of a user. The optical module has a housing with a pair of optical assemblies mounted therein. Each optical assembly comprises an eyepiece having an eyepiece optical axis. A beamsplitter is positioned along the eyepiece optical axis to form two optical channels—an eye tracking channel and a display channel. In an embodiment, the eye tracking channel is behind the beam splitter and directly in line with the optical axis of the eye tracking channel. The display channel is off axis to the eyepiece optical channel, such as substantially 90 degrees. In an embodiment, the axis of the optical channel extends substantially horizontally relative to the headset frame and substantially normal to the eyepiece optical axis. In other embodiments, the axis of the optical channel could be angled relative to the horizontal and/or eyepiece optical axis, such as to adjust the headset center of gravity. In this configuration, the beam splitter is configured to pass IR light and reflect visible light. In an alternative embodiment, the position of the two optical channels can be switched.

The eye tracking channel comprises an infrared (IR) imager and a camera lens configured to focus IR light onto an imaging plane of the imager. An IR illuminator, which can be positioned at the front of the eyepiece, is designed to emit IR light towards the eye of the user looking into the eyepiece. The IR light will reflect off of the eye and into the eyepiece, pass through the beamsplitter, and be focused by the camera lens to form an image of the user's eye on the imager.

The display channel comprises an electronic display, such as an LCD, that can generate a visible image, and adjustable display optics that are configured to relay an image output on the display for viewing through the eyepiece by a user looking through the eyepiece. The adjustable display optics can comprise a liquid lens with a spherical diopter that can be controlled electronically, such as across a range +10 to −10 diopters or other diopter range as appropriate for the use to which the system is to be put, and a display lens assembly that relays an intermediate image of an image on the display. The liquid lens, which may be one or more than one controllable lens, may also have adjustable cylindrical power to simulate or adjust for astigmatism. A variable iris diaphragm can be part of the display optics and placed, an embodiment, between the liquid lens and the relay display lens assembly. In an embodiment, the diaphragm is controlled by a motor to allow for automated adjustment.

Each optical assembly can be is mounted so that when the user is wearing the headset frame the front of each of the eyepieces is aligned with the user's eyes. In an embodiment, the distance between the optical assemblies are along an adjustment axis to allow of an intraocular distance between the eyepieces. In an embodiment, the optical assemblies are slidably mounted to a rotatable shaft. Threads on the shaft engage a threaded nut portion on each optical assembly so that rotating the shaft moves the optical assembly. The threads on the shaft where one optical assembly connects are opposite to where the other optical assembly connects so that the optical assemblies can be moved synchronously towards or away from each other to adjust the intraocular distance. A motor can be provided to draft the shaft or a manual control provided

In an embodiment, the eyepiece optical axes in the optical assembles are slightly angled away from each other, such as between 1 and 2 degrees although greater angles could be used. The angle can be selected to allow the forward ends of the two eyepieces to contact or nearly contact each other at the smallest intraocular distance while allowing a gap between other parts of the optical assembly, such as the channel in line with the eyepiece optical axis.

In an embodiment the IR illuminator comprises a ring of IR emitters mounted at or near the front of the respective eyepiece, such as surrounding the forward lens of the eyepiece. Because the diameter of the IR emitter ring may exceed that of the eyepiece lens, the emitter ring could limit the minimum intraocular distance. To allow for a smaller intraocular distance, each IR can have a circumferential gap positioned along the ring so that the gaps in each ring are opposed to each other. The gaps can be sized and positioned so that the structure of the IR emitter ring on the eyepieces do not limit the minimum intraocular distance.

The optical assemblies in the optical module can be controlled by a computer system to vary the stimulus shown on the displays, vary the power of the liquid lens, and perform eye tracking and imaging of the eyes of a user. The system can be used to perform eye examinations, such as visual field testing or as a substitute for a mechanical phoropter. Other visual testing applications are also possible.

### DESCRIPTION OF THE DRAWINGS

Further features and advantages, as well as structure and operation of various embodiments are disclosed in detail below with references to the accompanying drawings in which:

FIG. 1 is a high level illustration of a system for visual testing;

FIG. 2 is a schematic diagram of an optical assembly in the system of FIG. 1;

FIG. 3A is a cross-sectional view of a particular embodiment of the optical assembly of FIG. 2;

FIG. 3B is a detail view of the display channel of the optical assembly of FIG. 3A;

FIG. 3C is a detail view of the eye tracking channel and eyepiece assembly of the optical assembly of FIG. 3A;

FIG. 3D illustrates a portion of the eyepiece of FIG. 3A further showing an IR illumination assembly;

FIG. 4 is a simplified schematic diagram showing an intraocular adjustment mechanism for the optical assemblies of FIG. 1;

FIG. 5A is a rear perspective view of a particular embodiment of the optical assembly shown in FIG. 3A;

FIGS. 5B and 5C are partial views of a pair of optical assemblies of FIG. 5A mounted to a face plate showing an intraocular adjustment mechanism;

FIG. 5D is a front view of a pair of optical assemblies of FIG. 5A mounted to a face plate with the eyepieces at a minimum ocular distance;

FIG. 6A and 6B shows embodiments of left and right optical assemblies mounted in an optics module coupled 130 for use with a wearable headset.

### DETAILED DESCRIPTION

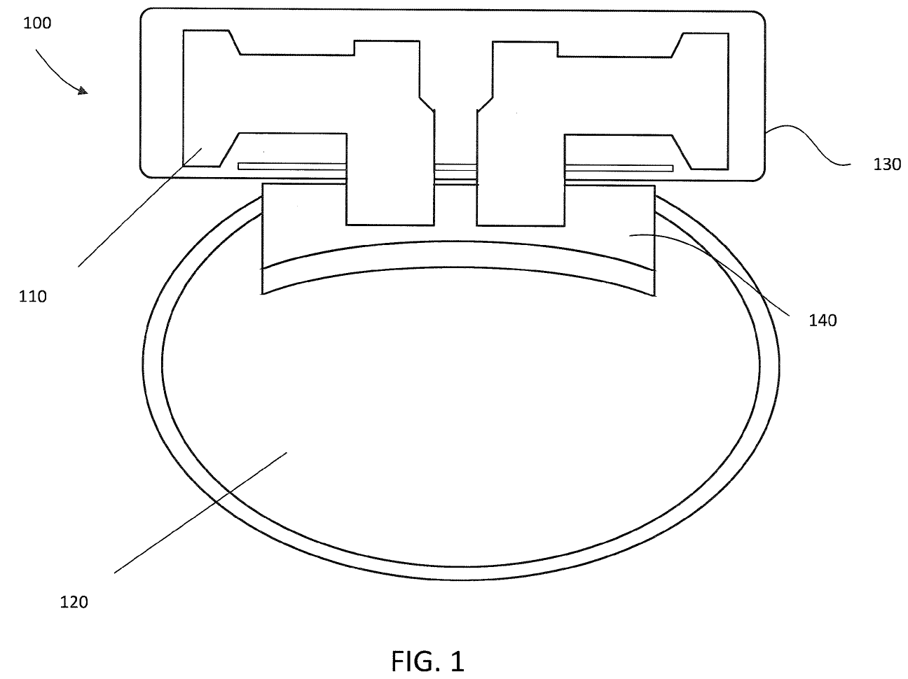

FIG. 1 is a high level illustration of a system 100 for visual testing. System 100 comprises a pair of optical assemblies 110, 110′, one for each eye, which can be mounted in a wearable headset 120. System 100 can be used to perform a variety of eye vision tests. In an embodiment, the optical assemblies 110, 110′ are mounted in an optics module 130. FIG. 1 shows both optical assemblies 110, 110′ within the same outer housing. Alternatively, each optical assembly 110, 110′ can be in a separate optics module 130, 130′, one for each eye, and each of which is connected to the headset frame 140. which can be removable coupled to a headset frame 140.

The optics module 130 can be can be integral with the headset frame 140. Alternatively, optics module 130 can be removably mounted to the headset frame 140. A particular headset configuration which can be adapted for this use is shown in U.S. Patent No. 11,504,000 entitled “Ophthalmologic Testing Systems and Methods”, the entire contents of which are expressly incorporated by reference. Various removable mounting mechanisms known to those of ordinary skill in the art can be used and will generally comprise interlocking male and female components on the optical module 130 and the headset frame 140. Mounting structures can include releasable spring or snap clips, a T-track style engagement, screw couplers, magnets, or other fasteners such as hook and loop material. Where dual optics modules 130/130′ are provided, each can be integral to or removably mounted to the headset frame 140.

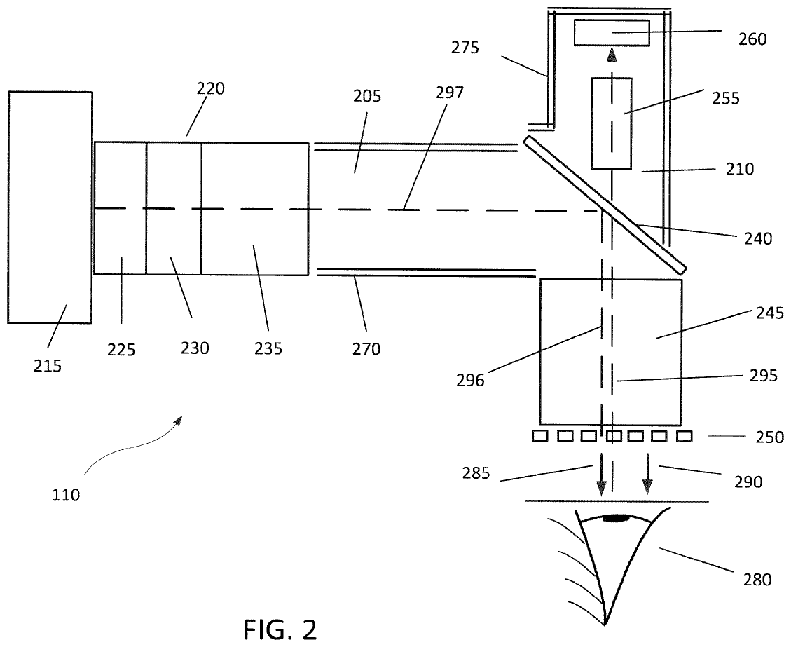

FIG. 2 is a schematic diagram of an optical assembly 110. Optical assembly 110 comprises a display channel 205, an eye tracking channel 210, and an eyepiece assembly 245 which is common to both channels 205, 210. The display channel 205 in optical assembly 110 can be several times longer than the housing 275 of the eye tracking channel 210. In an embodiment, the optical axis 295 of the eye tracking channel 210 is substantially collinear with the optical axis 296 of the eyepiece assembly 245 and the optical axis 297 of the display channel 205 is angled to that of the eyepiece assembly 245 and redirected using a beam splitter 240.

Display channel 205 comprises a display 215 which can be controlled to produce a visual image and adjustable display optics 220 which are used, in conjunction with an eyepiece 245 to provide an image to the eye 280 of a viewer. Display 215 can be a conventional flat panel display, such as an LED, LCD, or OLED display, with VGA, SVGA, XGA, or other resolution. The display should be small enough to allow for mounting within a headset and have a resolution sufficient to allow the desired optical testing to be done. In an embodiment, display 215 is a color active matrix TFT-LCD panel with an XGA resolution (1024×768) and has a display area of about 73 mm horizontal and 55 mm vertical. Other displays known to those of ordinary skill in the art can be alternatively used. Other display systems, such as a digital micro mirror device, could be used in alternative embodiments.

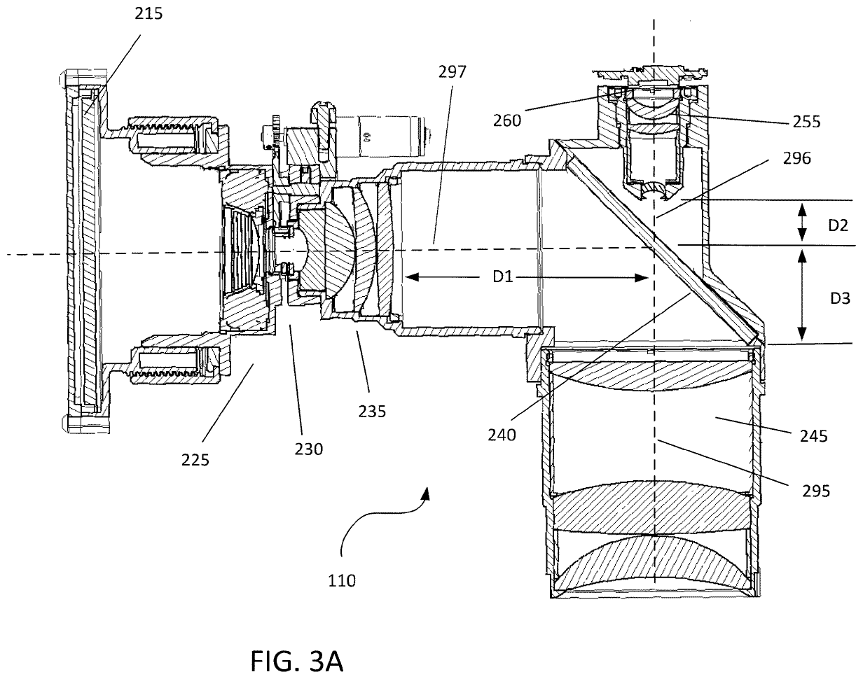

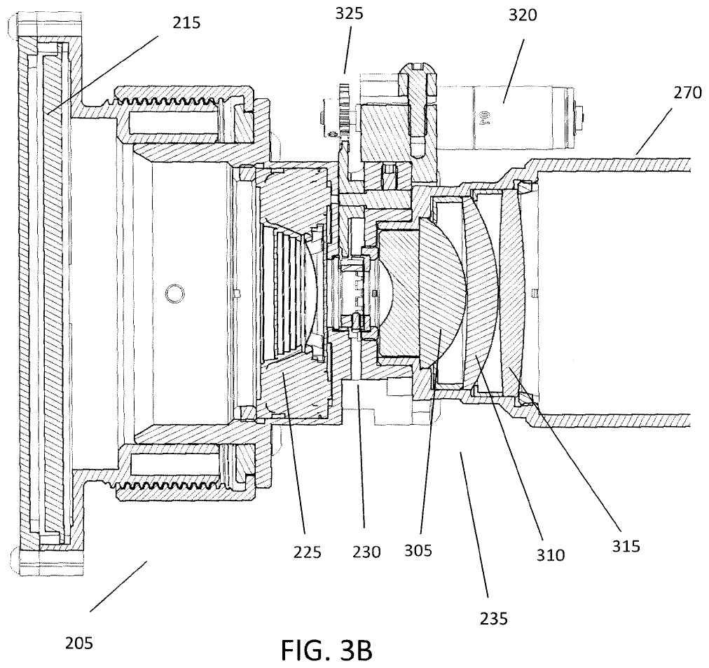

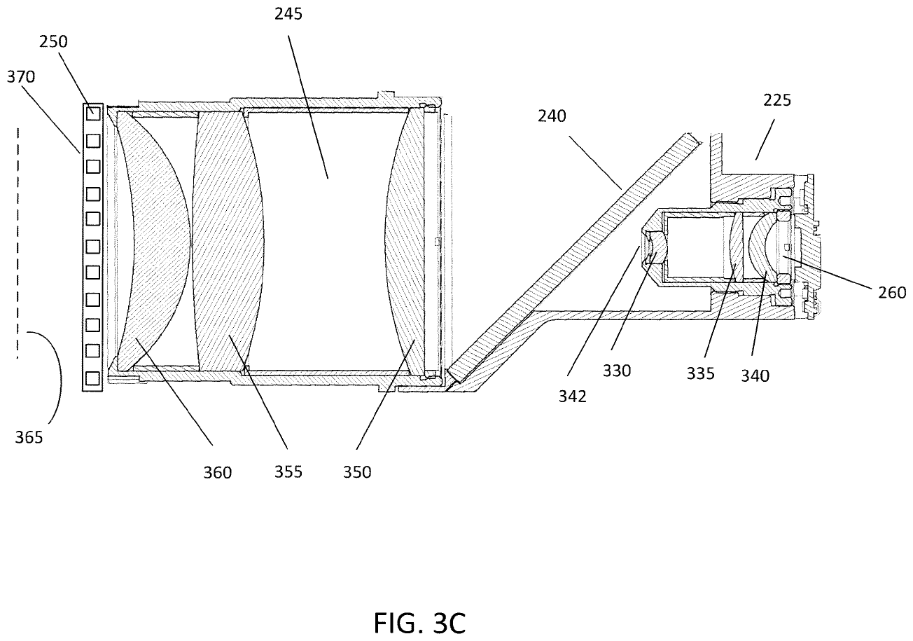

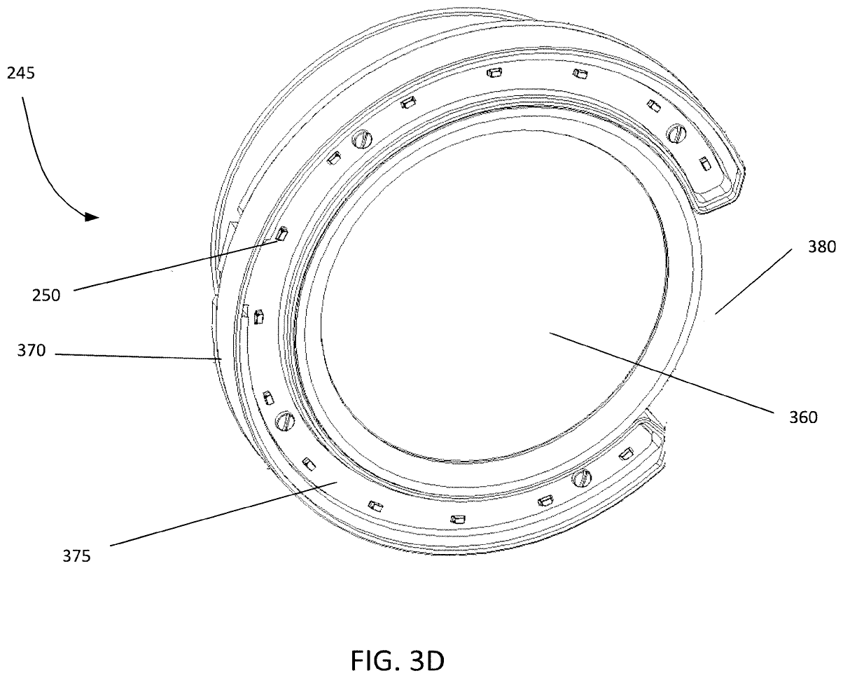

Adjustable display optics 220 operates as a relay that transmits the image formed at the LCD screen placed in the object plane into the plane of the eyepiece, forming the intermediate image. This lens system comprises a liquid lens 225 and a display lens assembly 235 and is designed for used with visual wavelengths produced by the display 215, such as the 486-656 nm wavelength band. An adjustable diaphragm 230, which can be motorized to allow for electronic control, can be included, such as between the liquid lens 225 and display lens assembly 235. Display optics 220 is discussed further below with respect to FIGS. 3A and 3B.

The liquid lens 225 can be electronically controlled to vary the spherical power of the lens 225 using conventional driving electronics. Varying the spherical power of the lens will change the distance to the intermediate image and, as a result, the apparent distance to the virtual image visible to the user changes. The liquid lens 225 may also have adjustable cylindrical power. Liquid lens 225 can be a single liquid lens or multiple separately controllable liquid lenses, such as a first liquid lens 225a used to adjust spherical power and a second liquid lens 225b used to adjust cylindrical power (not shown).

In operation, an image is generated on the display 215. The image light is focused by the adjustable display optics 220, and is redirected by the beam splitter 240 through the eyepiece 245 to produce a visual image 285. The spherical power of the liquid lens 225 can be adjusted to vary the apparent distance of the image shown on the display. The cylindrical power of the liquid lens 225 can be adjusted to compensate for (or introduce) astigmatism. In an embodiment, the spherical power of the liquid lens 225 can be varied across a range of at least ±10 diopters in the visible range of wavelength. A suitable lens is the Optotune model EL-16-40-TC. In alternative embodiments, the range can be at least ±15 diopters or ±20 diopters. The spherical diopter range should substantially accommodate the range of spherical anomalies in the human eye anticipated for users of the system 100. Similarly, in an embodiment where the liquid lens 225 provides cylindrical correction, the cylinder power should have a range of variability that substantially accommodates the range of cylindrical anomalies in the human eye anticipated for users of the system 100, such as at least ±6 diopters.

The diaphragm 230 can be adjusted to control the exit pupil diameter. The adjustments also can be used to vary the amount of light that is passed to the user's eye 280. Conventional motorizable iris assemblies can be used. In an embodiment, the diaphragm 230 has a minimum aperture range of less than 1 mm, such as 0.9 mm. In an alternative embodiment, the diaphragm size is not movable. Instead, the exit pupil size of the diaphragm is fixed and irradiance can e controlled by software which adjusts the brightness of the image emitted from the display.

As a user looking through the eyepiece is viewing an image produced by the display 215, their gaze direction can be tracked using an eye tracking camera system within the eye tracking channel 210. The camera system can also be used to capture continuous images of the user's eye for other purposes, such as, for example, to determine when the user's eye is open or closed or to measure the eye dilation. The eye tracking channel 210 comprises an infrared (IR) camera 260 and a camera lens assembly 255 which is configured to focus incoming infrared (IR) light onto the imaging plane of the camera 260. IR illumination of a user's eye is provided by one or more IR illuminators 250 that produce IR light 290 having a wavelength that is not visible to the human eye, such as between 810 nm and 850 nm. Reflected IR light from the eye 280 passes through the eyepiece 245 and at least a portion continues through the beam splitter 240 to enter the camera lens assembly 255 which focuses it into the IR camera 260. The beam splitter 240 can be a partially silvered mirror, a prism, or other design known to those of ordinary skill in the art. The beam splitter 240 used should be able to redirect visible light from the display to the eyepiece while passing the infrared light reflected from the eye to the eye tracking channel 210. In particular, in this configuration, beamsplitter 240 reflects radiation in the 480-660 nm wavelength band when installed at substantially a 45-degree angle and transmits radiation in the 800-890 nm wavelength band to allow for the combination of a visual optical channel and an IR eye-tracking optical channel as shown.

The IR illuminators 250 can be placed in a variety positions so long as sufficient IR light 290 reaches the user's eye with an adequate field of illumination for the eye tracking camera to be able to resolve the user's eye with sufficient clarity and resolution for eye tracking and imaging purposes. In a particular embodiment, and as discussed further herein, the illuminators 250 can be positioned in front of the eye pupil plane and provide an irradiance of approximately 250 W/m2. However, the specific illumination required is dependent, at least in part, on the sensitivity of the camera 260 and so different minimum values may be appropriate.

In the illustrated embodiment the display channel 205 is substantially normal, e.g., substantially 90 degrees, to the optical axis of the eyepiece 295, while the optical axis 296 of the eye tracking channel 210 is substantially collinear with eyepiece optical axis 295. Advantageously, this arrangement allows components of the camera lens assembly to be placed close to the beam splitter 240 without interfering with the reflected image from the display channel 205 and allows for a larger field of view in the eye tracking camera.

Putting the display channel off-axis from the eyepiece assembly 245 also advantageously allows the length of the optical assembly 110 along the optical axis of the eyepiece to be much shorter than the width of the optical assembly 110 normal to the optical axis of the eyepiece. This form factor advantageously allows the pair of optical assemblies 110 to be integrated with a wearable headset 120 so that the bulk of the weight positioned to the left and right of the eyepieces instead of extending outward from the eyepieces and the user's face. As a result, the center of mass of the optical assembly 110 remains close to the user's face so that the overall weight of system 100 is largely downward and strain on a user's neck is reduced. Notwithstanding, in an alternative embodiment the two optical channels could be changed so that the display optical channel 297 is collinear with the eyepiece optical axis 295 while the eye tracking channel is off axis.

While the optical axis 297 is illustrated as extending to the left and right of the eyepieces at substantially zero degrees to the horizontal, the angular orientation can be varied. For example, the optical channel 205 can be configured so its axis 297 extends substantially vertically at 90 degrees to the horizontal, at substantially 60, 45, or 30 degrees, or at another angle. These variations can allow for different headset form factors and can alter the position of the system center of gravity to provide for a more comfortable viewing.

The angle of the display channel 205 relative to the optical axis 295 of the eyepiece can also be varied. For example, the display channel 205 can be angled backwards (towards a user's head), by 5 degrees, 10 degrees, or 15 degrees from normal to the eyepiece optical axis, or by some other amount. This further angling can shift the center of mass of the system 100 towards the head of a user and can make the headset more comfortable to wear. Such a backwards angled configuration may be more suitable in a configuration of the system 100 in which the optical assembly 110 is an integral part of the headset frame 140 as this provides a greater range of options in where the straps used to hold the headset on the head of a user can be mounted. Varying the geometry may alter the reflection angle of the beam splitter 240. In a particular embodiment, the angle of the optical axis 297 of the display channel 205 is selected relative to horizontal and/or with respect to eyepiece optical axis 295 to place the center of gravity of the system within the vertical ‘shadow’ of the headset frame.

FIG. 3A is a cross-sectional view of a particular embodiment of the optical assembly 110. FIG. 3B is a detail view of the display channel 205. FIG. 3C is a detailed view of the eye tracking channel 210 and the eyepiece assembly 245. The optical properties of liquid lens 225 and display lens 235 are selected to provide a resolution to match the resolution of display 215 without substantial distortion and also to pair with the optical characteristics of the eyepiece 245. The eyepiece 245 is configured to minimize distortions of the image from the visual channel 205 and also to minimize reflections of IR light. The diaphragm 230 can be adjusted in a variety of ways. In this embodiment a stepper motor 320 drives a gear assembly 325 operative to open and close the diaphragm.

The display lens 235 in this embodiment is comprised of a spherical doublet lens 305, spherical biconvex lens 310 and a spherical concavo-convex lens 315.

The camera lens assembly 255 is configured to focus IR light reflected by the user's eye and that passes through eyepiece 245 onto the IR camera 260. The optical properties of the camera lens assembly 255 are selected so that IR light reflected from the field of view within which a user's eye 280 may present is focused across the imaging surface of the IR camera 260.

In an embodiment, the camera lens assembly 255 comprises a spherical concavo-convex lens 340, plano-convex lens 335 and convex-concavo lens 330. A leading aperture 342 can also be provided. If an IR pass filter is not integrated with the IR camera 260, one can be included as well to block stray visible light.

The eyepiece 245 operates to transmit the intermediate image formed in its object plane by the display lens assembly 230 Relay lens into the user's eye. The image of the iris diaphragm that is placed in the display lens assembly 230 is formed by the eyepiece in the exit pupil plane. The user's eye should be placed in the exit pupil plane to see the image. The eyepiece 245 also operates to transfer IR light reflected from the user's eye into the camera lens assembly 255 which projects the user's eye image into the camera 260. The eyepiece 246 is configured to minimize reflections from the IR illumination as well as distortions of the image from the display 215. In this embodiment, eyepiece 245 is comprised of a plano convex lens 350 for collimation, a biconvex lens 355, and a concavo-convex lens 360 as illustrated. An eyepiece cup (not shown) can be provided to help the user place the position their eye in front of the eyepiece and to block stray light.

The IR illuminators 250 can be situated inside the end of the eyepiece 245 in front of the eye pupil plane 265. The IR illuminators 250 can be IR LEDs and can be arranged in a circumferential LED ring 370 at the end of the eyepiece 245, such as adjacent the outermost lens and spaced from the eye pupil plane 265. The LED ring 370 can be positioned within a reflective ring 375 that directs the IR light outwards from the eyepiece in order to improve illumination efficiency. The distance from the LED ring 370 and the eye pupil plane 265 is dependent on the particular optical characteristics of the system, such as eye relief. In an embodiment, this distance can be approximately 17.5 mm. While LEDs are illustrated, other sources of IR light could be used instead, such as diffused light from an IR laser.

FIG. 3D is an illustration of the outer portion of eyepiece 245 illustrating lens 360 and the LED ring 370 within the reflective ring 375 in a particular embodiment. While LED ring 370 is shown as a circular element, other configurations are also possible. In an embodiment, and as discussed further below, the LED ring 370 and reflective ring 375 may extend around an arc that is not entirely circumferential. Instead, there is a gap 380 on the side of the eyepiece which would be adjacent another eyepiece when assembly 110 is used in a dual headset configuration, such as shown in FIG. 1. These opposed gaps allows for closer minimum spacing between left and right eyepieces when a pair of optical assemblies 110 are adjacent each other in embodiments configured as discussed further herein. The angular gap size can be selected based on the diameter of the ring 370 relative to the diameter of the eyepiece. In a particular embodiments, the gap angle is from between +/−20 to +/−50 degrees above a horizontal axis positioned between the centers of the eyepieces, between +/−35 to +/−45, and substantially 40 degrees,

In a specific embodiment, the entire display channel 205 is configured with an effective focal length from substantially 33.9 mm to 34.1 mm at 632.8 nm depending on liquid lens focal length. The lens configuration can allow for a visual field of 63° to 73° per eye piece. The motorized iris diaphragm controls the optical quality for different pupil sizes. IR illumination optics 370, 375 can be configured to provide a transverse width field of view for the eye tracking camera of 20 mm or more, such as 21 mm or 25 mm.

In one configuration for this specific embodiment, in the display lens assembly 235 doublet lens 305 has a focal length of substantially −41.05 mm at 632.8 nm, biconvex lens 310 has a focal length of substantially 49.52 mm at 632.8 nm, and lens 315 has a focal length of substantially 102.80 mm at 632.8 nm. In the eyepiece 245, lens 350 has a focal length of substantially 105.75 mm at 632.8 nm, lens 355 has a focal length of substantially 89.66 mm at 632.8 nm, and lens 360 has a focal length of substantially f=63.79 mm at 632.8 nm. In the camera lens assembly 330 has a focal length of substantially 80.47 mm @ 632.8 nm, lens 335 has a focal length of substantially 19.78 mm @ 632.8 nm, and lens 340 has a focal length of substantially 104.70 mm @ 632.8 nm. In this specific embodiment, The display lens assembly 235 is a distance D1 from the beamsplitter 240, camera lens assembly 255 is a distance D2 from beamsplitter 240, and eyepiece 245 is a distance D3 from beamsplitter 240. Measuring along the central optical axes, in this configuration D1 is substantially 63.15 mm, D2 is substantially 10 mm, and D3 is substantially 28.15 mm.

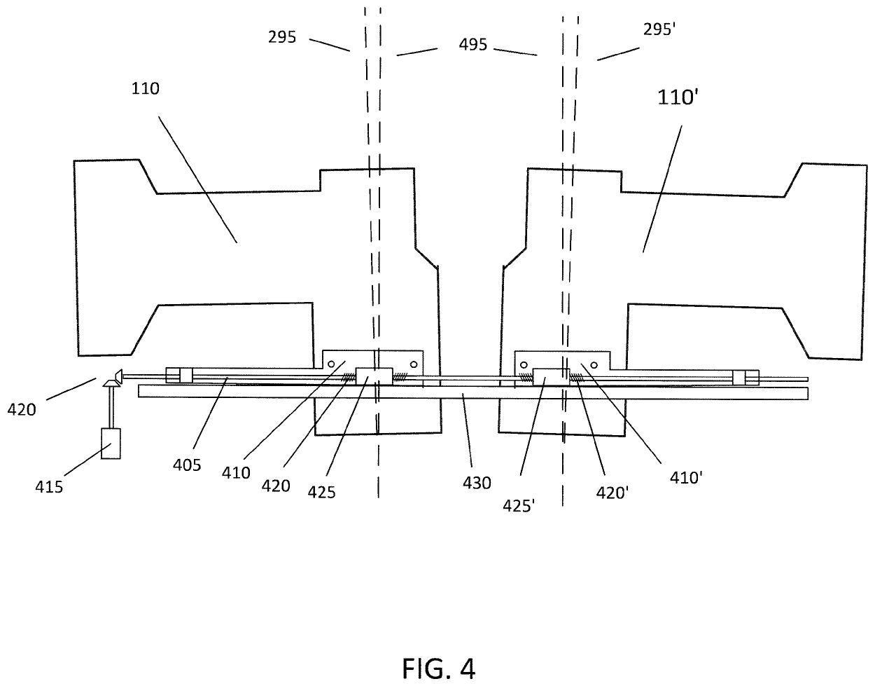

As discussed with respect to FIG. 1, left and right the optical assemblies 110, 110′ can be mounted in an optics module 130 that is part of a wearable headset 120. This configuration is shown in simplified schematic form in FIG. 4. Turning to FIG. 4, the optical assemblies 110, 110′ can be mounted to allow intraocular distance between the left and right eyepieces to be adjusted by adjusting the lateral position of the optical assemblies 110, 110′ along a common lateral adjustment axis, such as a shaft 405 and which is generally normal to the optical axis 295 of each eyepiece. While adjustment can be manual, the left and right optical assemblies 110 can be mounted to allow for a motorized adjustment. Other lateral adjustable mounting mechanisms known to those oFf skill in the art, such as a track mount, could be used instead of a shaft 405.

Various linear actuator mechanisms can be used to adjust intraocular distance. In an embodiment, each of the left and right optical assemblies 110 is coupled to a face plate 430 by respective left and right brackets 410, 410′. A common shaft 405 has left and right threaded portions 420, 420′ that passes through left and right threaded nut portions 425, 425′, respectively, and which are attached to the respective left and right brackets 410, 410′. The shaft 405 is driven by a motor 415, either directly or through a gearing assembly 420 as shown. Rotating the shaft 405 will cause the optical assemblies 110, 110′ to move relative to the face plate 430. As will be appreciated, the thread direction of the threaded portion 420 and nut portion 425 should be opposite to that of threaded portion 420′ and nut portion 425′ so that rotation of the shaft will cause the left and right optical assemblies 110, 110′ to move symmetrically towards or away from each other. Alternatively, instead of a motor 415, a manual adjustment mechanism, such as an externally accessible wheel, can be used to allow a user or operator to directly adjust the intraocular spacing.

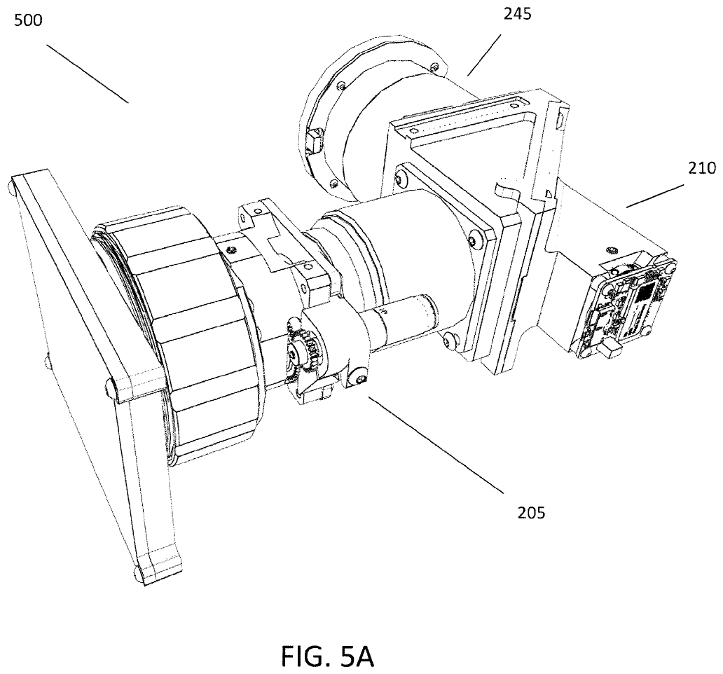

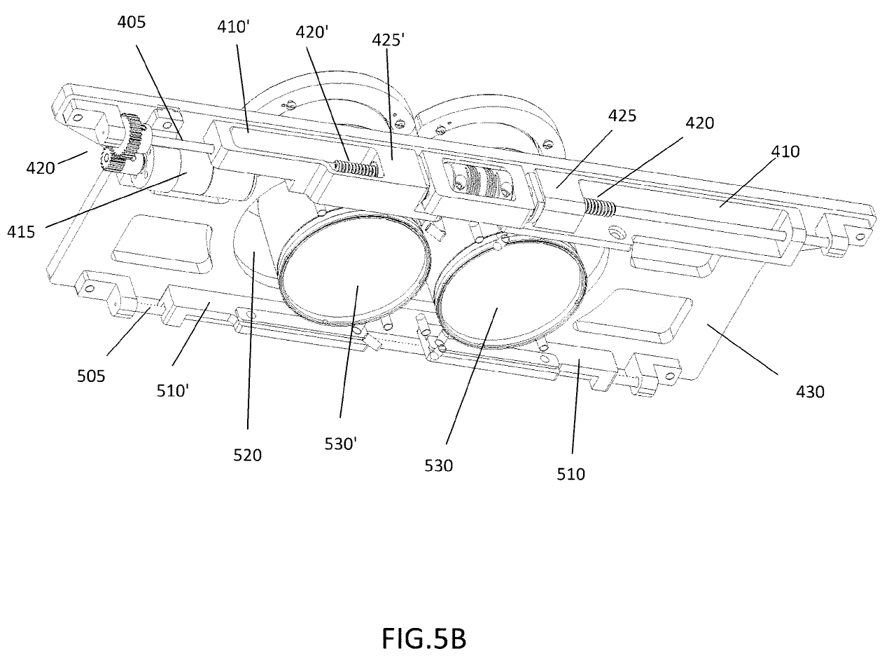

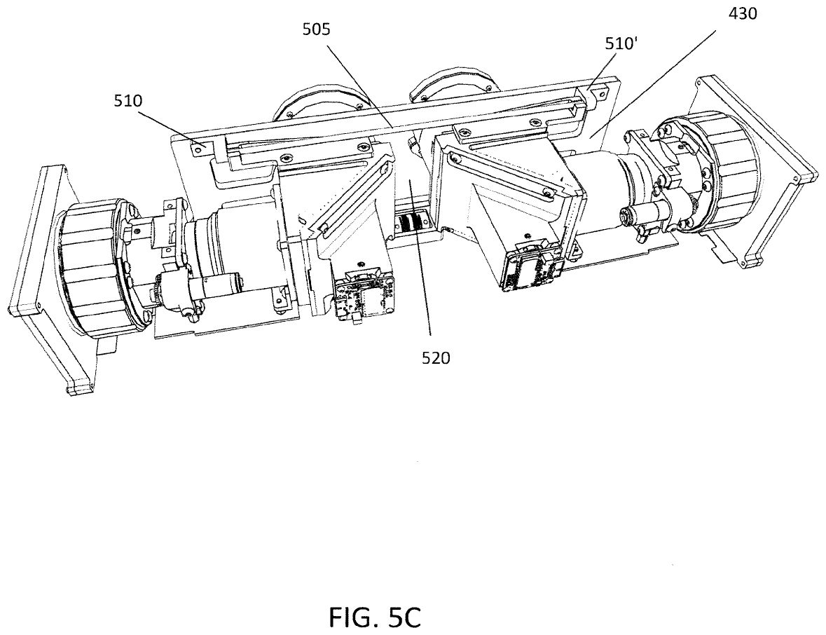

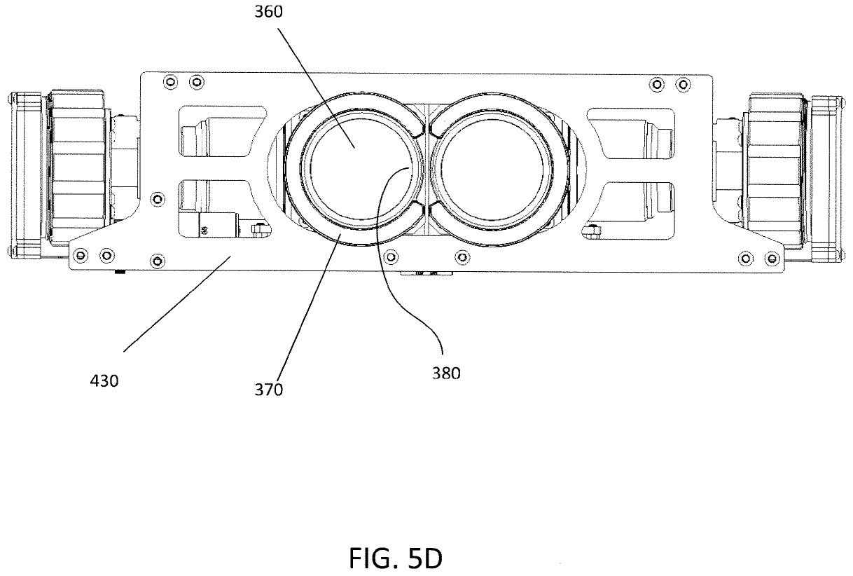

In an embodiment, the optical axis 295 of the eye tracking channel 210 in optical assembly 110 is angled away from the optical axis 295′ of the optical assembly 110′ by a small offset, such as between 1 and 2 degrees although greater angles such as up to 3 degrees, up to 5 degrees, or greater could be used. In FIG. 4 this angle is illustrated with respect to an axis 495 normal to the adjustment axis, which lies in this configuration along the shaft 405. In an embodiment, the offset of each optical assembly relative to axis 495 is substantially 1 degree. This angular offset allows the eyepieces in optical assemblies 110, 110′ to be positioned immediately adjacent each other while avoiding a collision between the outer housing of the assemblies 110, 110. The minimum distance between the eyepieces is further reduced because of the gap 380 in the LED illumination ring 370 as illustrated in FIG. 5D. Since the minimum eyepiece spacing is governed by the size of the lenses in the eyepiece and the eyepiece barrel, a large offset is not necessary to allow the ends of the closest to the user to be brought in substantial contact with each other even where the housings of assemblies 110, 110′ along the eyepiece optical axis greater inward width than the eyepieces do.

FIG. 5A is a rear perspective view of a particular embodiment 500 of the optical assembly 110 shown in FIG. 3A. FIGS. 5B and 5C are a partial top and bottom perspective views, respectively, of the embodiment of FIG. 5A illustrating an embodiment of the intraocular adjustment mechanism of FIG. 4. FIG. 5D is a front view of a pair of optical assemblies of FIG. 5A mounted to a face plate and showing the eyepieces adjusted to a minimum ocular distance. As illustrated in FIGS. 5B and 5C, shaft 405 connects the left and right eye channel pieces 530, 530′ across the top of the face plate 430 and is controlled by motor 415 coupled to the shaft 405 by gears 420. A second shaft 505 can be provided to connects the two eye channel pieces 530, 530′ along the bottom of the face plate 430 via respective brackets 510, 510′. The shaft 505 can be undriven. Alternatively, the shaft 505 can be driven by the motor 415 using a conventional coupling mechanism (not shown).

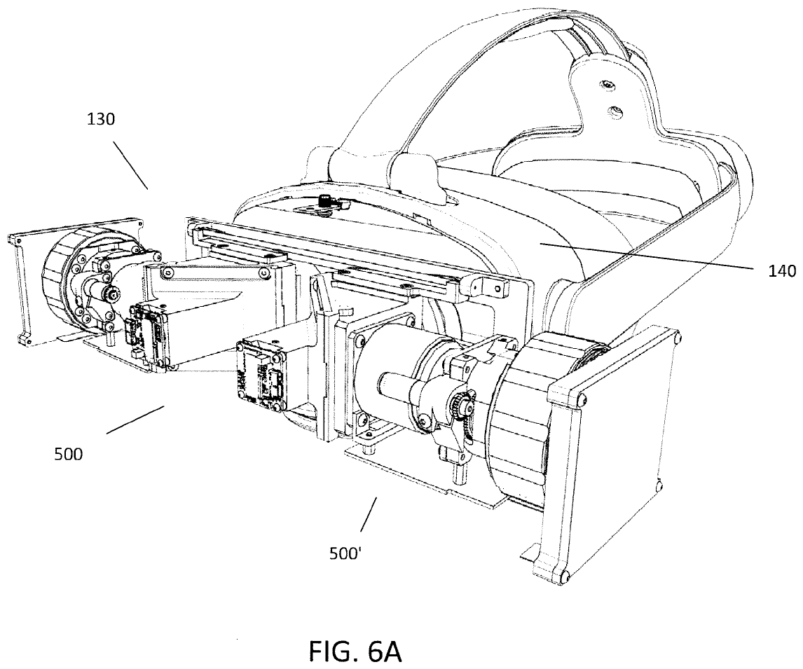

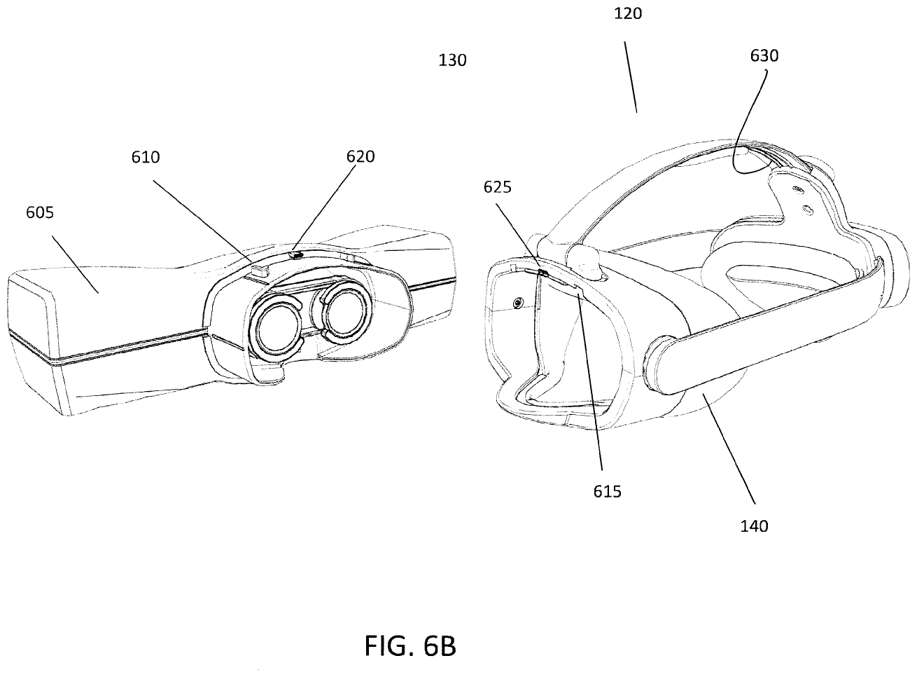

FIG. 6A is an illustration of left and right optical assemblies 500, 500′ as shown in FIG. 5A mounted together to form an embodiment of an optics module 130 coupled to a headset frame 140 of a wearable headset 120. In FIG. 6A an outer housing is omitted. FIG. 6B is an embodiment of the optics module 130 of FIG. 6A within a module housing 605 and where the optics module 130 is configured to be removably mounted to the headset frame 140 of a wearable headset 120. Male and female interlocking components 610, 615 on the optics module 130 and headset frame 140 can be used to secure the optics module 130 in place. Male and female electrical couplers 620, 625 can be provided to connect the electrical components in optics module 130 to a cable 630 in the headset 120 through which power and control signals can be provided to the optics module 130 and data retrieved. Conventional electronic interfaces and power couplings can be provided to drive the display 215, adjust the liquid lens 225, diaphragm 230, intraocular spacing, control the IR camera 260 and illuminators degree and read image data captured by the camera. Additional features of an optics module that is removably coupled to a headset are disclosed in incorporated by reference U.S. Pat. No. 11,504,000.

System 100 can be configured to provide an enhanced visual field using light weight components to allow for a larger than conventional visual field in a portable medical device. Liquid lens technology allows for spherical and cylindrical power correction via current-driving electronics. Stereographic projections can be generated to produce three dimensional simulated environments.

In a particular application, system 100 can be used for visual field testing. A computer system connected to system 100 (not shown) can generate test images which are output on one or both of the displays. Data from the eye tracking camera can be used to monitor the gaze direction of the user to help determine when features in the test image are or are not visible. The images from the eye tracking camera can also be used to determine the pupil diameter of the user. This value can be used by the computer to control the size of the diaphragm and thereby the exit pupil of the optical system to ensure that the optical quality of the image being seen by the patient from has the highest quality possible.

In a visual field testing process using system 100, a computer running testing software (not shown) can be coupled to the headset electronics and display test images one or both of the displays. For example, the liquid lens can be set to provide a virtual distance from the image equal to a standard distance, such as 30 cm. A stimulus sequence can then be shown on one or both displays. For example, dots that appear at various locations on a circular grid within the visual field of the display can be shown. As stimulus is presented, the user can indicate whether or not they see the stimulus. If a point is not seen by a patient, the user can continue to be tested on the rest of the test points needed. After the set of stimulus has been presented, an array of visual field points that marks where a patient did not see the stimulus can be generated. The missed test points can also be tested once again one. In an embodiment of this process, the size of the stimulus (Goldmann size) can be increased by one size relative to the prior (unseen) stimulus. This process can continue until the stimulus is detected or the maximum Size of Goldman Size V has been reached. The eye tracking camera can be used to monitor the gaze direction of the user to ensure that the patient is looking straight ahead when visual field data is collected in during the testing. When the user is not looking straight ahead, the relative position of the stimulus within the user's visual field can still be determined by adjusting according to the determined direction of gaze.

A further application is use of the system 100 as a substitute for a mechanical phoropter system and physical eye charts or wall projections. Stereographic rendering of letters can be shown in a virtual environment on one or both of the displays in system 100. A doctor or other person giving the eye test can control the spherical and cylindrical power of the liquid lens through a computer interface instead of having to rotate a set of dials and flip lenses a mechanical phoropter. An initial prescription can be input using the computer and the software will interpret it to provide the appropriate control signals to changes the state of the liquid lens to reflect that prescription.

Other uses for system 100 will be appreciated by those of ordinary skill in the art. Various aspects, embodiments, and examples of the invention have been disclosed and described herein. Modifications, additions and alterations may be made by one skilled in the art without departing from the spirit and scope of the invention as defined in the appended claims

1. An optical system comprising:
a headset frame configured to be worn on the face of a user, the headset frame having a front and a back, the back adjacent a face of the user when the headset frame is worn;
first and second optical assemblies, each respective optical assembly comprising:
an eyepiece having an eyepiece optical axis and a front;
an IR illuminator configured to emit IR light towards the eye of the user looking into the eyepiece, wherein IR light will reflect off of the eye and into the eyepiece;
a beamsplitter positioned along the eyepiece optical axis and having an eye tracking channel and a display channel in optical communication therewith;
the eye tracking channel having an eye tracking optical axis and comprising an infrared (IR) imager and a camera lens configured to focus IR light from the eyepiece onto an imaging plane of the imager;
the display channel having a display channel optical axis and comprising an electronic display and adjustable display optics configured to relay an image output on the display for viewing through the eyepiece by a user looking through the eyepiece;
wherein one of the eye tracking optical axis and the display channel optical axis is substantially collinear with the eyepiece optical axis;
the first optical assembly and second optical assembly mounted to the headset frame so that when the user is wearing the headset frame the front of the eyepiece in the first optical assembly is generally aligned with a first eye of the user and the front of the eyepiece in the second optical assembly is generally aligned with a second eye of the user.

2. The system of a claim 1, wherein a distance between the first optical assembly and the second optical assembly is adjustable along an adjustment axis to thereby allow adjustment of an intraocular distance between the front of the eyepiece of first optical assembly and the front of the eyepiece of the second optical assembly.

3. The system of claim 2, further comprising;
a faceplate;
a rotatable shaft coupled to the faceplate and having first and second threaded portions therein, a thread direction of the first threaded portion being opposite a thread direction of the second threaded portion;
the first optical assembly having a first threaded nut portion engaged with the first threaded portion of the shaft;
the second optical assembly having a second threaded nut portion engaged with the second threaded portion of the shaft; and
a motor coupled to the shaft;
wherein rotation of the shaft changes the intraocular distance.

4. The system of claim 2, each of the eyepiece optical axis of the first optical assembly and the eyepiece optical axis of the second optical assembly having an angle relative to the adjustment axis of between substantially 0.5 and substantially 5 degrees.

5. The system of claim 4, wherein each of the eyepiece optical axis of the first optical assembly and the eyepiece optical axis of the second optical assembly is angled at substantially 1 degree relative to the adjustment axis.

6. The system of claim 4, wherein the IR illuminator of each respective optical assembly comprises a respective ring of IR emitters at the front of the respective eyepiece.

7. The system of claim 6, wherein each respective ring has a circumferential gap, the gap in the ring in the first optical assembly and the gap in the ring in the second optical assembly opposed to each other.

8. The system of claim 7 wherein the gap is substantially 40 degrees.

9. The system of 8, wherein the adjustable display optics in each respective optical assembly comprises a respective liquid lens with an electronically controlled spherical diopter power and a respective display lens assembly.

10. The system of claim 9, the adjustable display optics in each respective optical assembly further comprising a respective adjustable diaphragm between the liquid lens and the respective display lens assembly.

11. The system of claim 9, wherein the spherical diopter of the liquid lens in each respective optical assembly is adjustable from between substantially −10 to substantially +10.

12. The system of claim 9, wherein the liquid lens in each respective optical assembly further has an electronically controlled cylindrical diopter power.

13. The system of claim 1, wherein the first optical assembly and the second optical assembly are inside a common housing that is removably coupled to the headset frame.

14. The system of claim 1, wherein for each respective optical assembly:
the respective eye tracking optical axis is substantially collinear with the respective eyepiece optical axis;
the respective beam splitter is configured to pass IR light from the respective eyepiece into the respective eye tracking channel and reflect visible light from the respective display channel towards the respective eyepiece.

15. The system of claim 14, wherein for each respective optical assembly the respective display channel optical axis is substantially normal to the respective eyepiece optical axis.

16. An optical system attachable to a headset frame configured to be worn on the face of a user, the optical system comprising;
a housing;
first and second optical assemblies within the housing, each respective optical assembly comprising:
an eyepiece having an eyepiece optical axis and a front;
an infrared (IR) illuminator at the front of the eyepiece and configured to emit IR light outwards from the eyepiece, wherein when a user is looking into the eyepiece with an eye, emitted IR light will reflect off of the eye and into the eyepiece;
a beamsplitter positioned along the eyepiece optical axis and having a front facing the eyepiece and a back;
an eye tracking channel and having an eye tracking optical axis substantially coaxial with the eyepiece optical channel and comprising an IR imager and a camera lens configured to focus IR light passing from the eyepiece and through the beamsplitter onto an imaging plane of the imager; and
a display channel having a display channel optical axis intersecting the beamsplitter and substantially normal to the eyepiece optical axis and comprising an electronic display and adjustable display optics configured to relay an image output on the display for viewing through the eyepiece by the user looking into the eyepiece;
the first and second optical assemblies movably mounted relative to each other within the housing along an adjustment axis to thereby allow adjustment of an intraocular distance between the front of the eyepiece of first optical assembly and the front of the eyepiece of the second optical assembly;
the housing configured to be removably mounted to the headset frame,
wherein when the housing is mounted to the headset frame and the user is wearing the headset frame a position of the front of the eyepiece in the first optical assembly relative to the headset frame and a position of the front of the eyepiece in the second optical assembly relative to the headset frame is adjustable so as to align the front of the eyepiece in the first optical assembly with a first eye of the user and to align the front of the eyepiece in the second optical assembly with a second eye of the user and
wherein the user can see images output on the respective electronic displays in the first and second optical assemblies and the first and second eyes of the user can be imaged by the respective IR imager in the first and second optical assemblies.

17. The system of claim 16, each of the eyepiece optical axis of the first optical assembly and the eyepiece optical axis of the second optical assembly having an angle relative to the adjustment axis of substantially 1 degree.

18. The system of claim 17 wherein the IR illuminator of each respective optical assembly comprises a respective ring of IR emitters having a circumferential gap, the gap in the ring in the first optical assembly and the gap in the ring in the second optical assembly opposed to each other.

19. The system of claim 16, the adjustable display optics in each respective optical assembly comprising:
a respective liquid lens with an electronically controlled spherical diopter power;
a respective display lens assembly;
and a respective adjustable diaphragm between the liquid lens and the respective display lens assembly.

20. The system of claim 19, further comprising:
a faceplate;
a rotatable shaft coupled to the faceplate and having first and second threaded portions therein, a thread direction of the first threaded portion being opposite a thread direction of the second threaded portion;
the first optical assembly having a first threaded nut portion engaged with the first threaded portion of the shaft;
the second optical assembly having a second threaded nut portion engaged with the second threaded portion of the shaft; and
a motor coupled to the shaft;
wherein rotation of the shaft changes the intraocular distance.
